(19) United States
(12) Patent Application Publication
(10) Pub. No.: US 2009/0269411 A1
(43) Pub. Date: Oct. 29, 2009

(54) MASKING THE TASTE OF POWDERS

(75) Inventors: Rainer Bellinghausen, Odenthal (DE); Daniel Rudhardt, Koln (DE); Frank Ridder, Kurten (DE); Martin Steinbeck, Koln (DE); Jesko Zank, Koln (DE); Martin Weiss, Bornheim/Sechtem (DE); Olaf Behrend, Koln (DE); Udo Van Stiphout, Moers (DE)

Correspondence Address: NORRIS, MCLAUGHLIN & MARCUS, P.A., 875 THIRD AVE, 18TH FLOOR, NEW YORK, NY, 10022, US

(73) Assignee: Bayer Technology Services GmbH, Leverkusen (DE)

(21) Appl. No.: 12/158,725

(22) PCT Filed: Dec. 20, 2006
(86) PCT No.: PCT/EP2006/012284

§ 371 Date: Nov. 17, 2008

(30) Foreign Application Priority Data
Dec. 24, 2005 (DE) 102005062270.4

(51) Int. Cl. A61K 9/14; B05D 3/00; A61P 35/00

(52) U.S. Cl. 424/490; 427/2.14

(57) ABSTRACT

Taste-masked powders that can be inhaled or administered orally, a simple method for the production thereof, and the use thereof for applying biologically active substances.

The present invention relates to novel taste-masked powders for inhalation or oral administration, a simple process for production thereof and use thereof for applying biologically active substances.

When bitter-tasting active compounds are inhaled, generally, poor taste occurs during or after the inhalation, which frequently leads to low acceptance of the inhalates on the part of their users. Therefore, masking or flavoring inhalable powders is desirable. Consumer compliance is increased which, in the case of oral formulations, is proven and has become thoroughly established.

Even if in the case of modern inhalation formulations the effective dose is extraordinarily high (>>90% of the active compound reaches the lungs), taste impairment cannot be avoided thereby. Human taste perception generally reacts to extremely small levels of contamination. Therefore, masking which does not effect the good level of activity of dry powder inhalers is a clear marketing advantage compared with formulations without taste masking.

The taste masking of inhalates described in the literature is restricted to the pulverization of aromas, such as, for example, WO2001/26630, WO93/17663, JP11-106339.

Encapsulation of relatively large bodies, for example tablets, is already known in principle. It is also known that microcapsules in the size range greater than 200 μm can be encapsulated in the fluidized bed, for example in what is termed Wurster coaters.

Smaller particle sizes can be coated by condensation encapsulation, in which case, however, a vaporizable coating material is required. (see: Ebert, Dau, “Beschichten submikroner Partikeln durch heterogene Kondensation unter Expansion” [Coating submicron particles by heterogeneous condensation with expansion], DFG-Jahresbericht 2003).

Encapsulation of powders for controlled release is described in “Controlled dissolution from wax-coated aerosol particles in canine lungs”, J. Appl. Physiol. 84(2), 1998, 717-725.

In addition, in DE 19753794, coatings of inhalable powder were used to improve the free-flowing quality, for example powders based on electrostatically charged casing material.

The conventional processes, however, are not usable for masking powders having particles sizes (d₅₀) of about 5 μm, since they lead to a thick coating layer. For example, in the coating of tablets, generally 2-10 mg of coating material/cm² are used, which corresponds to layer thicknesses of 20-100 μm. A process for encapsulating powders that are to be inhaled, however, must only build up very thin coating layers, since otherwise the aerodynamic diameters of the particles are changed too greatly and the encapsulated powder is then no longer suitable for inhalation. The aerodynamic diameter of a particle in this case is defined as the diameter of a sphere having the normalized density of 1 g/cm³ which has the same falling velocity as the particles themselves.

At the same time, the thin coating layers must, however, lead to a tight envelopment which does not permit a release until after a time of 15-30 min, since otherwise the desired taste masking is not ensured.

Other very recently developed encapsulation methods such as co-grinding or centrifugal fluidized beds show either poor taste masking or problems, for example, in the case of hygroscopic materials (citric acid) which have a tendency to agglomerate, as a result of which the encapsulated powders can no longer be processed.

There was therefore a requirement for a process for producing taste-masked inhalable powders by encapsulation which leads to a thin but also tight coating layer and is simple and inexpensive to carry out.

It has now surprisingly been found that this object is achieved by a process comprising the distribution of a pulverulent solid having a median particle diameter d₅₀ of 1 to 40 μm, preferably 2 to 10 μm, particularly preferably approximately 4 to 6 μm, in a solution of a hydrophobic coating agent in a solvent which does not dissolve the pulverulent solid and then lowering the temperature of the resultant mixture to precipitate out the coated solid and if appropriate isolating the coated solid. In this case the fraction of the coating agent can be varied. The preferred range is considered to be 50 to 99% by weight (based on the sum of pulverulent solid and coating agent), such that for the individual particle size ranges layer thicknesses of the coating agent of 1 to less than 20 μm, preferably 1 to 5 μm, and particularly preferably 1 to 3 μm, are obtained.

The process according to the invention is suitable in principle for all types of pulverulent solids. Preferably, these are active compounds, that is to say substances from the group of agents for healing, alleviation or prevention of disorders of humans or animals such as, for example, acidosis therapeutics, analeptics/antihypoxamatics, analgesics/antirheumatics, anthelminthics, antiallergics, antiaenemics, antiarrhythmics, antibiotics/antiinfectives, antidementives, antidiabetics, antidotes, antiemetics/antivertigo agents, antiepileptics, antihemorrhagics, antihypertonics, antihypoglycemics, antihypotonics, anticoagulants, antimycotics, antiparasitic agents, antiprotozoics, antiphlogistics, antitussives/expectorants, arteriosclerosis agents, broncholytics/antiasthmatics, cholagogues and bile duct remedies, cholinergics, corticoids, dermatics, diuretics, blood circulation stimulants, withdrawal agents/agents for treating addictive diseases, enzyme inhibitors, preparations for enzyme deficiency and transport proteins, fibrinolytics, geriatric remedies, antigout agents, gynecological remedies, hepatics, hypnotics/sedatives, immune modulators, cardiac agents, coronary agents, laxatives, lipid lowering agents, local anesthetics/neural therapeutics, gastrointestinal tract remedies, migraine agents, muscle relaxants, ophthalmics, osteoporosis agents/calcium metabolism regulators, otologic agents, psychopharmaceuticals, rhinological agents/sinusitis agents, roborants/tonics, thyroid therapeutics, sex hormones and their inhibitors, spasmolytics/anticholinergic agents, thrombocyte aggregation inhibitors, tuberculosis agents, natural immune modulation agents, urologics, venous therapeutics, vitamins, cytostatic agents, other antineoplastic agents and protective agents.

Examples which may be mentioned in this context are boldin, quinolones, ciprofloxacin, felodipine, flurbiprofen, ibuprofen, ketoprofen, macrolides, nicardipine, nifedipine, nimodipine, nisoldipine, nitrendipine, norfloxacin, moxifloxacin, ofloxacin, paclitaxel, praziquantel, sulfonamides and tetracyclines.

The coating material is a hydrophobic water-repellant material. Hydrophobic in the context of this invention is also taken to mean materials which are insoluble or water-soluble only with restrictions. The coating material must be virtually insoluble at a temperature of 25° C. in water at pH 6 to 7.5, or at least <1000 mg/kg soluble. Such hydrophobic materials can be:
Waxes having a melting range of 30-180° C. such as paraffins, natural waxes, beeswaxes, carnauba wax, saturated hydrocarbons of the form CnH2n+2, synthetic waxes, Fischer-Tropsch waxes, stearines, macrogol stearate, and chemically modified wax types, vinyl polymers, montan ester waxes and montan wax fatty acids.
Resins: petrochemical-origin hydrocarbon resins, polymers of unsaturated aromatic C₉-C₁₀-hydrocarbons with and without phenol, aliphatically modified aromatic C₉-C₁₀-hydrocarbons having an unsaturated aliphatic component, indene-coumarone resins, polymers of carbochemical unsaturated aromatic hydrocarbons, phenol-modified indene-coumarone resin, copolymer of carbochemical unsaturated C₉-C₁₀-hydrocarbons with phenol,
Polymethacrylates and copolymers thereof
Polylactides and polylactide glycolide copolymers
Chitosan, natural products from chitin-containing natural substances and chemical modifications thereof
Water insoluble polyether compounds, polyether polysulfone
Chemically modified cellulose derivatives, their acetates, succinates, sulfonates having water-insoluble properties as described above.

Examples of such hydrophobic coating agents are carnauba wax from Baerlocher GmbH and also waxes from Sasol Wax GmbH, for example types 5203, 4110, 6202, 6805, C80 and C100, resins and novolac from the companies RÜTGERS Chemicals AG and Ashland-Südchemie-Kernfest GmbH, Eudragite, in particular the E types E100 and EPO, from Degussa Röhm, chitosan from Cognis, hydroxypropylmethyl-celluloseacetatesuccinate (AQCOAT) from Shin-Etsu AQOAT.

Suitable solvents for carrying out the process according to the invention are, for example, aromatic or aliphatic hydrocarbons which are liquid at room temperature, in particular linear or cyclic alkanes which can if appropriate be branched. Likewise suitable are organic solvents, in particular one selected from the group of short-chain alcohols having 1 to 10 carbon atoms, such as, for example, methanol, ethanol, 2-propanol, the short-chain glycols, such as, for example, ethylene glycol, 1,2-propylene glycol, the short-chain ketones having 3 to 10 carbon atoms, such as, for example, acetone, 2-butanone, carboxylic acids such as, for example, acetic acid, ethers, such as, for example, diethyl ether, tetrahydrofuran or methyl tert-butyl ether, esters such as, for example, methyl acetate, ethyl acetate or methyl formate, heterocyclic amines such as, for example, pyridines, formamides such as, for example, dimethyl formamide, or else n-methylpyrrolidone or dimethyl sulfoxide. Particularly preferred solvents are n-heptane and methylcyclohexane. The above-mentioned solvents can in each case be use alone or in a mixture.

After production of a mixture of pulverulent solid, solvent and coating agent, the coated solid is formed by lowering the temperature (cold precipitation). Typically, the production of said mixture proceeds at a temperature of 50° C., preferably 40 to 100° C.

To carry out the cold precipitation, in the second step, conventionally cooling is performed to a temperature of 20° C., preferably 0 to 40° C.

The concentration of the coating agent in the solvent is conventionally about 5 to 25%, depending on the solubility, also above or below. Saturated solutions should be employed. The fraction of the pulverulent solid of said mixture is generally 1 to 90%, preferably 5 to 20%.

The coated solid, after it has been formed, is isolated by known methods, for example by spray drying.

The coated solid particles produced by the process according to the invention surprisingly have only a very thin coating layer, so that the particle size and in particular the aerodynamic diameter are scarcely altered. Nevertheless, these coated solid particles exhibit successful taste masking. The coated solid particles produced by the process according to the invention are therefore ideally suitable for use in dry powder inhalers and oral dosage forms which also require efficient taste masking on biting or chewing.

The small particle size, in addition, in the case of the oral dosage form, prevents the capsules from being bitten open on chewing. This is particularly advantageous in applications as chewing tablets and also in the case of medicaments for animals and children.

A further advantage on oral application is the improved mouthfeel, since the small particles are not perceived as particles.

The invention will be illustrated by the examples hereinafter, but without being restricted thereby.

### EXAMPLES

### Example 1

### (Praziquantel with Wax C80)

2.8 g of ground Praziquantel having a particle size of <10 μm (particle size distribution after encapsulation: d90=9.0 μm; d10=1.5 μm, solid dispersed in Myritol, 120″ ultrasound, Malvern Master Sizer, lens 100 mm) were stirred at 70° C. into a solution of 22.2 g of wax C80 (commercially available from Sasol Wax GmbH) in 200 g of heptane. Subsequently, the temperature of the resultant mixture was cooled to 20° C. at a cooling rate of 10 K/h with stirring using a Mizer disc of diameter 57 mm at 500 rpm and the capsules formed were isolated by spray drying in a Buechy-laboratory spray dryer using a pneumatic nozzle of diameter 0.5 mm with an input air temperature of 140° C. and an outlet air temperature of 80° C.

The particle sizes of the encapsulated Praziquantel are in the range of approximately 2-9 μm (d10 and d90, see above). Taste tests show that the bitter taste, after application of the formulation to the tongue, is not noticed even after a period of 10 minutes. Even chewing the formulation over a plurality of minutes does not lead to release of the taste.

### Example 2a to d

### (Ciprofloxacin with Carnauba Wax)

Here also ground active compound is stirred into a wax solution and the temperature is lowered so that the wax precipitates out. Isolation proceeded again by spray drying.

The active compound content was varied between 5 and 20%:

Ground ciprofloxacin having a particle size of 0.5 to 9 μm (d10 and d90 in Q3 distribution) were stirred into a solution of carnauba wax (commercially available from Baerlocher GmbH) in said proportions (based on the coating agent) at 60° C. Subsequently the temperature of the resultant mixture was cooled to 20° C. at a cooling rate of 10 K/h with constant stirring using an impeller of diameter 60 mm at 450 rpm and the capsules formed were isolated by spray drying in a Buechy-laboratory spray dryer, in a similar manner to Example 1.
2a: 342 g of methylcyclohexane, 38 g of carnauba wax, 2 g of ciprofloxacin
2b: 100 g of methylcyclohexane, 28 g of carnauba wax, 7 g of ciprofloxacin
2c: 303 g of heptane, 30 g of carnauba wax, 1.6 g of ciprofloxacin
2d: 152 g of heptane, 15 g of carnauba wax, 3.8 g of ciprofloxacin

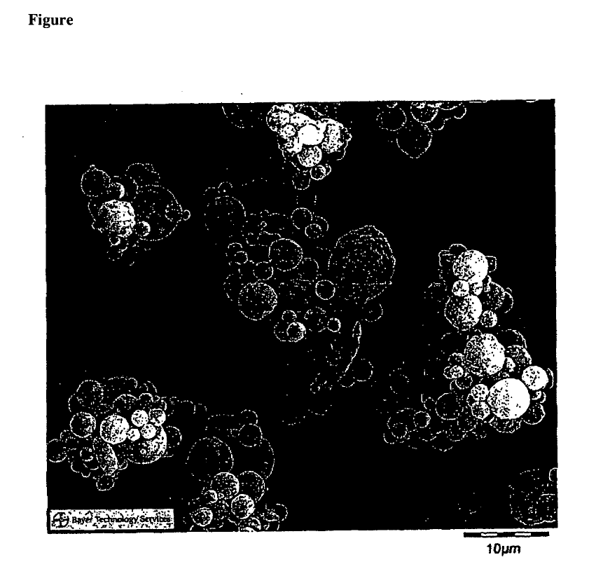

An REM image of the capsules obtained in Example 2a is presented as FIG. 1. The successful taste masking was established as follows: the coated material was placed onto the tongue and flushed off after approximately 10 min. The strongly bitter taste of the active compound was not noticed. For comparison, pure active compound was also tested: the bitter taste occurred very rapidly and the taste test had to be terminated prematurely.

### Example 3

### (Not According to the Invention)

Using the known processes, coacervates of praziquantel with the familiar encapsulating agents gelatin and CMC were produced and cured. However, these had more rapid release in water than the uncoated active compound, and no taste masking could be achieved.

1. A coated solid comprising a pulverulent solid having a particle diameter of 1 to 40 μm, and a coating of a hydrophobic coating material having a fraction of 50 to 99% by weight (based on the sum of pulverulent solid and coating agent).

2. The coated solid as claimed in claim 1, wherein the coating agent has a thickness ranging from 1 to less than 20 μm.

3. The coated solid as claimed in claim 1, wherein the pulverulent solid is an active compound.

4. A process for producing coated solids as claimed in claim 1, comprising a process comprising distributing a pulverulent solid having a median particle diameter d₅₀ of 1 to 40 μm in a solution of a hydrophobic coating agent in a solvent which does not dissolve the pulverulent solid and then lowering the temperature of the resultant mixture to precipitate out the coated solid and optionally isolating the coated solid.

5. The process as claimed in claim 4, Wherein the pulverulent solid is an active compound.

6. The process as claimed in claim 4, wherein the hydrophobic coating agent is a wax having a melting point of 30-100° C.

7. The process as claimed in claim 4, wherein the solvent is heptane or methylcyclohexane.

8. The process as claimed in claim 4, wherein the production of the mixture proceeds at about 60° C. and the mixture is subsequently cooled to about 20° C.

9. The process as claimed in claim 4, wherein the isolation of the coated solid proceeds by spray drying.

10. A powder inhalate or an oral dosage from comprising a coated solid as claimed in claim 1.
